(19) United States
(12) Patent Application Publication
(10) Pub. No.: US 2009/0250841 A1
(43) Pub. Date: Oct. 8, 2009

(54) Tracheal tubes and a method of making the same

(75) Inventor: Daniel Jay Coates, Ogden Dunes, IN (US)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 12/078,920

(22) Filed: Apr. 8, 2008

(51) Int. Cl. A61M 16/04; B29C 45/00

(52) U.S. Cl. 264/328.1; 128/207.15

(57) ABSTRACT

A tracheal tube is made by the attachment proximate to its patient end an inverted cuff, so that the flash lines that result from the molding of the cuff is hidden from view, and any loose material from the flash lines of the cuff is trapped within the cuff, instead of falling into the patient. The smooth surface of the inverted cuff also prevents potential secretion buildup on the cuff and facilitates the cleaning of the tracheal tube.

### FIELD OF THE INVENTION

The present invention relates to tracheal tubes and in particular to a tracheostomy tube with an inverted cuff.

### BACKGROUND OF THE INVENTION

In the making of cuffed tracheal tubes, for example tracheostomy tubes, and in particular those tracheostomy tubes that are made from silicone, the silicone cuffs that are attached to those tubes are usually formed by injection molding where a mold consists of an upper cover and a lower base. As a result, there are often flash lines or parting lines formed along the lengths of the cuffs where the upper portion of the mold meets the lower portion of the mold. The flash line results from excess silicone that seeps out where the upper and lower portions of the mold meet. Although efforts have been made as part of the manufacturing process to eliminate the flash lines at the cuffs, the unsightly flash lines nonetheless are readily seen and may be felt by the patient or be snagged when the tracheal tube is inserted into the patient. Another problem that may occur is that the flash or excess material that form the flash lines may fall off while the tracheal tube is in the patient.

### SUMMARY OF THE PRESENT INVENTION

To minimize discomfort to the patient and also provide an appearance of smoothness, the present invention cuffed tracheal tube is made with the cuff inverted or turned inside-out so that the smooth interior surface of the cuff is exposed to the environment while the original exterior surface of the cuff with the flash lines is attached, at its end collars, to the distal end of the tubular shaft of the tracheal tube. By inverting the cuff, the flash line(s) that formed at the outside surface of the cuff is hidden from view and also will not cause discomfort to the patient as the tracheal tube is inserted to the patient. Moreover, any flash or excess material that may detach from the cuff at the flash line is trapped within the cuff, and therefore not fall into the patient. Furthermore, by having a completely smooth outer cuff surface, upon inflation, a better seal is formed between the cuff and the trachea of the patient. Also, the smooth cuff surface prevents potential secretion buildup on the cuff. So, too, a smooth cuff surface eases or facilitates the cleaning of the tracheal tube.

The present invention therefore relates to a tracheal tube that includes a tubular shaft, a cuff including collars one at each of its ends having its original inner surface inverted so that its original outer surface at the two collars are attached proximate to a patient end of the shaft at spaced locations. A lumen is integrally formed along at least a portion of the shaft with its patient end located inside the cuff for enabling a fluid, such as for example air or a liquid, to be input from the machine end of the lumen into the cuff to inflate the cuff.

The present invention is also related to a tracheostomy tube that includes a silicone tubular shaft that has a patient end and a machine end whereat an inflation lumen is attached, a flange integrally formed as part of the shaft at the machine end of the shaft, and a silicone cuff including collars one each of its ends having its original inner surface inverted so that its original outer surface at the two collars are attached proximate to the patient end of the shaft at spaced locations and its original inner surface being exposed to the environment. The lumen has a portion thereof formed integrally along a substantial length of the shaft with its patient end located inside the cuff so that a fluid may be input to the cuff to inflate the cuff.

The present invention moreover is related to a method of manufacturing a tracheal tube that includes the steps of: molding a tubular shaft, molding a cuff including collars one at each of its ends, inverting the cuff so that its original inner surface becomes the surface that is exposed to the environment and its original outer surface forms the interior of the cuff, attaching the original outer surface of the two collars of the cuff proximate to the patient end of the shaft at spaced locations, and integrally forming a lumen along at least a portion of the shaft with its patient end located inside the cuff to enable the cuff to be inflated when a fluid is input to the machine end of the lumen.

### BRIEF DESCRIPTION OF THE FIGURES

The present invention will become more apparent and the invention itself will be best understood by reference to the following description of the invention taken in conjunction with the following drawings, wherein:

FIG. 1 is a side view of a cuff for a tracheal tube after it has been removed from its mold;

FIG. 2 is a cross-section view of the FIG. 1 cuff showing the interior surface of the cuff;

FIG. 3 is a perspective view of the cuff shown in FIGS. 1 and 2 having been inverted so that the original inside surface is now exposed to the environment;

FIG. 4 is a side view of a tracheal tube, for example a tracheostomy tube of the present invention;

FIG. 5 is a top view of the tracheostomy tube of FIG. 4;

FIG. 6 is a top view of the lower half of a mold with a core used for making a plurality of cuffs of the instant invention positioned therein;

FIG. 7 is a side view of the lower portion of the mold as shown in FIG. 6 relative to its upper portion; and

FIG. 8 is a perspective view of the lower portion of the mold of FIG. 6.

### DETAILED DESCRIPTION OF THE INVENTION

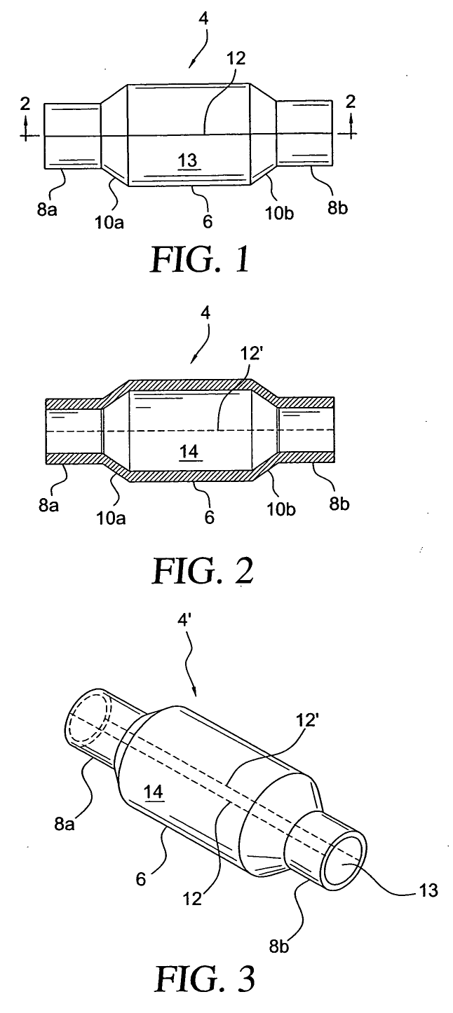

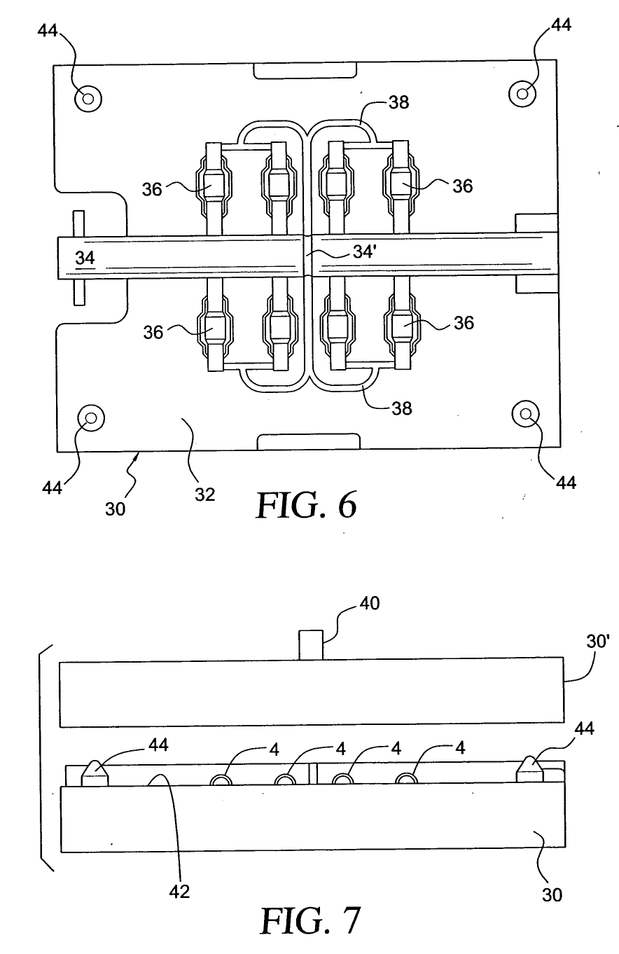

With reference to FIG. 1, a cuff 4 made from injection molding is shown. The cuff may be made of silicone rubber or other material such as PVC and has an inflatable center portion 6 and two collars 8a and 8b at its ends. Collars 8a and 8b extend from main body portion 6 via respective transition portions 10a and 10b. When removed from the mold, flash lines are usually formed longitudinally along opposite sides at the outer surface of cuff 4 due to the molding flash or excess material attached to the cuff at the junction or plane where the upper and lower portions of the mold meet, as will be further explained with respect to FIGS. 6-8. For the embodiment where the cuff is molded from silicone, the excess material is silicone.

Attempts have been made to remove this excess material, or molding flash. But in the case of a silicone cuff, due to its thin layer and soft texture, short of tearing or causing a hole, there remains at the cuff at least a portion of the flash line 12 that could not be removed. The flash line opposite flash line 12 in the back of cuff 4 shown in FIG. 1 is shown as a dotted line 12′ in the cross-sectional view of cuff 4 in FIG. 2. The exterior or outer surface of cuff 4 is designated 13 in FIG. 1.

With reference to FIG. 2, the cross-sectional view 2--2 of cuff 4 is shown. As was noted earlier, the flash line 12′ on the exterior surface 13 of cuff 4 away from the reader is designated 12′. Further as shown, the interior or inside surface 14 of cuff 4 is smooth and unmarked by any defect from the molding process.

FIG. 3 is a perspective view of the cuff shown in FIG. 1 having been inverted or turned inside-out, so that its original inner surface 14 is now exposed to the environment while its original outer surface 13 is now the inner surface of the cuff. For ease of discussion, the cuff shown in FIG. 3 is designed 4′. All other designations remain the same as those in FIG. 1. As shown, flash lines 12 and 12′ are now within the interior surface of cuff 4′, so that the exterior surface 14 of cuff 4′ is now completely smooth.

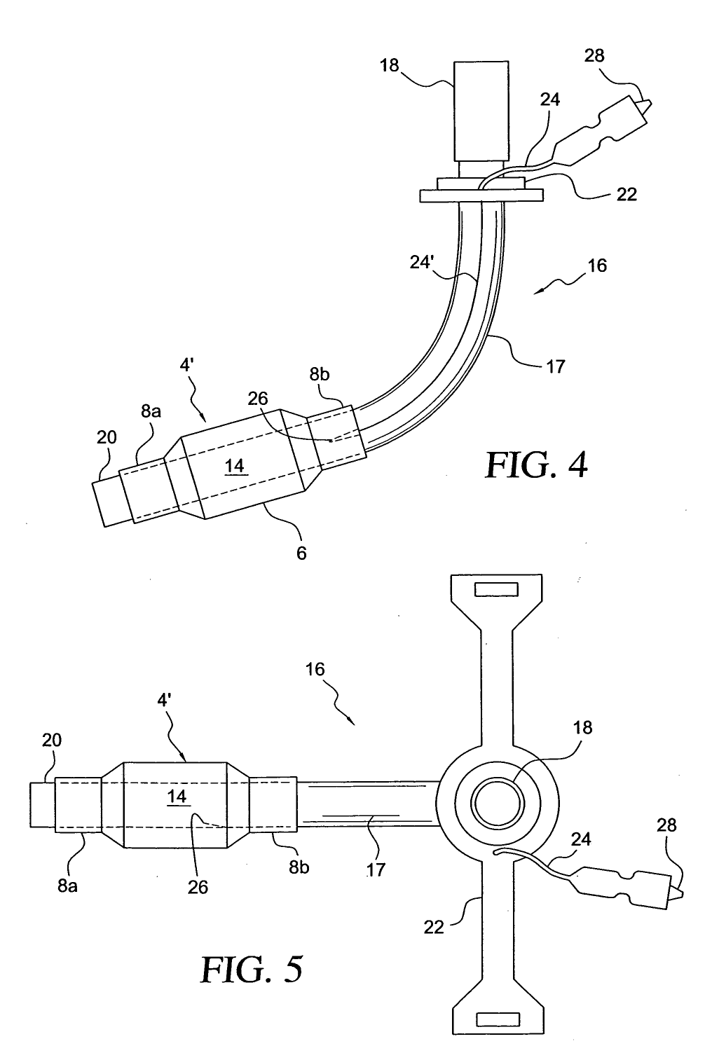

FIG. 4 shows an exemplar tracheal tube, for example a tracheostomy tube 16. Tracheostomy tube 16 is formed by an elongate tubular shaft 17 that has a patient end 20 and a machine end 18. Shaft 17 may be a straight tube, or may be curved along a part of its length. Proximate to the patient end 20, cuff 4′ is attached by means of its glue bands or collars 8a and 8b at spaced locations proximate to the patient end 20. The tracheostomy tube for this invention is preferably made also from silicone and in the preferred embodiment is injection molded as a single integral unit to have a flange 22, best shown in FIG. 5, that allows the tracheotomy tube 16 to be attached to the patient. For this embodiment of tracheostomy tube 16, the inverted cuff 4′ is a TTS (Tight To Shaft) fit to shaft 17, where the cuff lies close to the shaft when it is in its non-inflated resting state. This minimizes any snag to the cuff when the tracheostomy tube is placed into a patient.

Attached to tracheostomy tube 16 at its machine end 18 close to flange 22 is a lumen or tube 24 that is integrated along a path 24′ substantially along the length of shaft 17 of the tracheostomy tube, with its patient end 26 residing within main portion 6 of cuff 4′. By connecting a syringe or other input means to its machine end 28, a fluid such as for example air or a liquid, possibly sterile water, may be fed through lumen 24 into cuff 4′ to inflate cuff 4′.

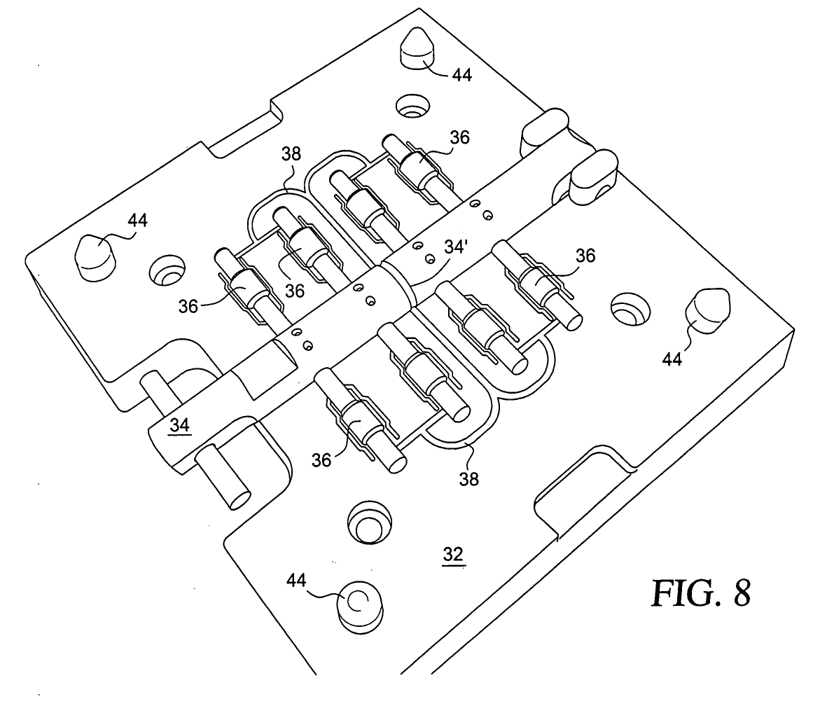

With reference FIGS. 6 and 8, the lower portion of the mold for making the cuff of the instant invention is shown. As illustrated, the lower mold 30 is made of either hardened steel or aluminum, or some metal alloy such as beryllium and copper. Mold 30 is shown to include a bed 32 onto which a central mold core 34 that has attached thereto a plurality of cuff-shaped branch cores 36 is positioned. Central core 34 is removable from bed 32 so that the silicone rubber cuffs formed on the cuff-shaped branch cores 36 may be extracted. As further shown, there is a groove 34′ formed at the center of core 34, and there are a number of grooves or runners 38 formed in bed 32 of mold 30. Runners 38 allow the silicone material, poured into the mold as a liquid resin from a sprue or input port 40 shown in the upper mold 30′ of FIG. 7, to flow around the cuff-shaped branch cores 36 to form the plurality of cuffs. This of course is done only after the upper mold 30′ has been placed over and secured to the lower mold 30. Upper mold 30′ is guided onto lower mold 30 by guide pins 44.

When the silicone liquid resin is injected into the mold, by way of sprue 40 at upper mold 30′, the liquid silicone resin first flows along groove 34′ at core 34 and then flows along the runners 38 to surround the various cuff-shaped branch cores 36. After curing, upper mold 30′ is lifted off the lower mold 30 and central core 34 is removed from lower mold 30. The individual cuffs formed at the branch cores 36 could then be removed. Note that since a mold is made up of upper and lower portions, upon the positioning of upper mold 30′ over the lower mold 30, during the injection and curing processes, there usually is formed a parting line at each side of each of the cuffs at the plane where the bottom surface of upper mold 30′ meets the upper surface of the lower mold 30. These parting lines result in the flash lines 12 and 12′ shown in FIGS. 1-3 and discussed above. The parting lines or flash lines for the different cuffs, designated 4, formed at the branch cores 36 are best shown in FIG. 7 along the plane 42.

By inverting the cuffs retrieved from the cuff-shaped branch cores 36, the smooth inner surface 14 is now exposed to the environment. By attaching cuff 4′ to shaft 17 at spaced locations with an adhesive, for example a liquid silicone RTV (Room Temperature Vulcanization), the flash lines that, prior to the instant invention would cause discomfort to the patient and render the tracheostomy tube unsightly, is hidden from view. Moreover, the flash, or excess silicone material, that could fall off from the flash line is now trapped within the interior of cuff 4′, more specifically the space defined by the interior surface of cuff 4′ and the outer surface of shaft 17 covered by cuff 4′. Further, the smooth cuff surface prevents potential secretion buildup on the cuff and facilitates the cleaning of the tracheostomy tube after its removal from the patient. Furthermore, the flash lines in the interior surface of cuff 4′, which due to the TTS configuration, may establish an air passage between the inner surface 13 of cuff 4′ and the outer surface of shaft 17, to thereby assist in the inflation of cuff 4′. In other words, as the cuff is almost a parallel sleeve to the shaft for a TTS type tracheostomy or tracheal tube, in the case where the cuff is not inverted, the smooth inner surface of the cuff may come into contact with and be stuck to the outer circumferential surface of the shaft so that inflation of the cuff may be more difficult.

While the present invention is disclosed with reference to tracheal or tracheostomy tubes and cuffs that are made from silicone, it should be appreciated that the instant invention is also applicable to other cuffed tracheal tubes made from materials that may also cause parting or flash lines for the cuffs. Moreover, instead of injection molding, the present invention cuff may be manufactured by other molding methods including for example blow molding.

1. A tracheal tube comprising: a tubular shaft, a cuff including collars one at each of its ends having its original inner surface inverted so that its original outer surface at said two collars are attached proximate to a patient end of said shaft at spaced locations, a lumen integrally formed along at least a portion of said shaft with its patient end located inside said cuff for enabling a fluid to be input from a machine end of the lumen into said cuff for inflating said cuff.

2. Tracheal tube of claim 1, wherein said cuff is made from silicone by injection molding.

3. Tracheal tube of claim 1, wherein said tubular shaft is made from silicone.

4. Tracheal tube of claim 1, further comprising a flange integrally formed as one piece with said shaft, the inflation lumen is also integrally formed along said shaft.

5. Tracheal tube of claim 1, wherein the original outer surface of said cuff comprises at least a portion of one flash line resulting from the molding of said cuff, and wherein the attaching of the original outer surface of said cuff to said shaft at spaced locations at the collars traps any material separating from the flash line within said cuff.

6. Tracheal tube of claim 1, wherein said fluid is air or a liquid.

7. A tracheostomy tube comprising: a silicone tubular shaft, said shaft having a patient end and a machine end whereat an inflation lumen is attached, a flange integrally formed as a part of said shaft at the machine end of said shaft, a silicone cuff including collars one at each of its ends having its original inner surface inverted so that its original outer surface at said two collars are attached proximate to the patient end of said shaft at spaced locations and its original inner surface is exposed to the environment, said lumen having a portion thereof formed integrally along a substantial length of said shaft with its patient end located inside said cuff so that a fluid may be input to said cuff to inflate said cuff.

8. Tracheostomy tube of claim 7, wherein said cuff is made from injection molding.

9. Tracheostomy tube of claim 7, wherein the original outer surface of said cuff comprises at least a portion of one flash line resulting from the molding of said cuff, and wherein the attaching of the original outer surface of said cuff to said shaft at spaced locations at the collars traps any material separating from the flash line within said cuff.

10. A method of manufacturing a tracheal tube, comprising the steps of:
molding a tubular shaft;
molding a cuff including collars one at each of its ends;
inverting said cuff so that its original inner surface becomes the surface that is exposed to the environment and its original outer surface forms the interior surface of said cuff;
attaching the original outer surface of the two collars of said cuff proximate to a patient end of said shaft at spaced locations; and
integrally forming a lumen along at least a portion of said shaft with its patient end located inside said cuff to enable said cuff to be inflated when a fluid is input to the machine end of said lumen.

11. Method of claim 10, wherein said cuff is injection molded from silicone.

12. Method of claim 10, wherein said shaft is injection molded from silicone.

13. Method of claim 10, wherein said cuff is attached to said shaft by the original outer surface at the collars being bonded to said shaft by a liquid silicone RTV (room temperature vulcanization) adhesive, the bonded cuff trapping material that may separate from a flash line formed at the original outer surface of said cuff.

14. Method of claim 10, further comprising the step of integrally molding a flange proximate to a machine end of said shaft so that said shaft and said flange are formed as a unitary single piece.
